(19) United States
(12) Patent Application Publication
(10) Pub. No.: US 2006/0014722 A1
(43) Pub. Date: Jan. 19, 2006

(54) Per (3,6-anhydro) cyclodextrin derivatives, their preparation and use thereof for separating ions, in particular chromium and manganese anions

(76) Inventor: Andree Gadelle, Montbonnot (FR)

Correspondence Address: FOLEY AND LARDNER LLP;SUITE 500, 3000 K STREET NW, WASHINGTON, DC, 20007, US

(21) Appl. No.: 10/517,582

(22) PCT Filed: Jun. 11, 2003
(86) PCT No.: PCT/FR03/01741

§ 371 Date: Aug. 1, 2005

(30) Foreign Application Priority Data
Jun. 12, 2002 (FR) 02/07205

(51) Int. Cl. A61K 31/728; C08B 37/16

(52) U.S. Cl. 514/58; 536/103

(57) ABSTRACT

The invention relates to per(3,6-anhydro)cyclodextrin derivatives containing in C₂ at least one carbamate group, polymers thereof, their preparation and their use for separating contaminating anions such as the anions CrO₄²⁻, Cr₂O₇²⁻ or MnO₄²⁻.

### TECHNICAL FIELD

The subject of the present invention is novel per(3,6-anhydro)cyclodextrin derivatives and polymers based on per(3,6-anhydro)cyclodextrins, which can be used in particular for binding and separating ions such as anions based on chromium and manganese.

This invention finds its application in the field of environmental decontamination in relation to these polluting ions, and for human decontamination.

### PRIOR STATE OF THE ART

Cyclodextrins or cyclomaltooligosaccharides are compounds of natural origin formed by the cyclic linkage of α-(1,4)-linked glucose units. Derivatives thereof may consist of α-(1,4)-linked maltose units.

Numerous studies have shown that these compounds could form inclusion complexes with hydrophobic molecules, thus allowing their solubilization in aqueous media. Numerous applications have been proposed in order to exploit this phenomenon, in particular in the pharmaceutical field, as is described by D. Duchêne “Pharmaceutical application of cyclodextrins” in “Cyclodextrins and their industrial uses”. D. Duchêne Ed., Editions de Santé, Paris, 1987, pages 213-257 [1].

Among the very many modified derivatives of these cyclodextrins, those for which the cavity is turned inside out exhibit advantageous properties even though their capacity to enclose organic molecules is lost or is very limited. Nevertheless, this capacity to enclose hydrophobic molecules can be recovered if the chain substituting the hydroxyl in C₂ of the cyclodextrin is longer. Compounds of this type are the per(3,6-anhydro)cyclodextrins.

The synthesis of these peranhydrocyclodextrins has been described since 1991 in document [2]: Gadelle A. and Defaye J., Angew. Chem. Int. Ed. Engl., (1991), 30, pages 78-79; and document [3]: Ashton P. R., Ellwood P., Staton I. and Stoddart J. F., Angew. Chem. Int. ed. Engl., (1991) 30, pages 80-81, and it has been shown that these derivatives have advantageous solubilities both in water and in organic solvents. Some subsequent studies (document [4]: Yamamura H. and Fujita K. Chem. Pharm. Bull., (1991) 39, pages 2505-2508; document [5]: Yamamura H., Ezuka T., Kawase Y., Kawai M., Butsugan Y. and Fujita K., J. Chem. Soc., Chem. Com., (1993), pages 636-637; and document [6]: Yamamura H. Nagaoka H., Kawai M. and Butsugan Y., Tetrahedron Lett. (1995) 36, pages 1093-1094) have furthermore shown that these peranhydro derivatives could complex alkali metal ions with a nonnegligible selectivity.

The document FR-A 2 744 124 [7], the document FR-A 2 764 525 [8] and the document FR-A 2 807 044 [9] mention other per(3,6-anhydro)cyclodextrin derivatives substituted at the 2-position, which are used for the separation of various ions, in particular potassium and caesium in the case of document [7] by virtue of the presence of the acetyl substituent, or lead in the case of document [8] by virtue of the presence of a methoxy substituent or polluting ions such as the cobalt or uranyl ion and the lanthanide ions in the case of document [9] by virtue of the presence of a substituent —O—CH₂—CO₂H.

Nevertheless, the derivatives described in these documents do not make it possible to bring about a satisfactory separation by complexing the anions based on chromium and manganese, which can pollute the environment.

### DISCLOSURE OF THE INVENTION

The subject of the present invention is precisely novel derivatives and novel polymers of peranhydrocyclodextrins in which the substituent at the 2-position has been chosen so as to confer on these compounds properties of complexing anions based on chromium or manganese, such as the chromate, bichromate and permanganate anion.

According to the invention, the per(3,6-anhydro)cyclodextrin derivative corresponds to one of the following formulae (I) or (II): 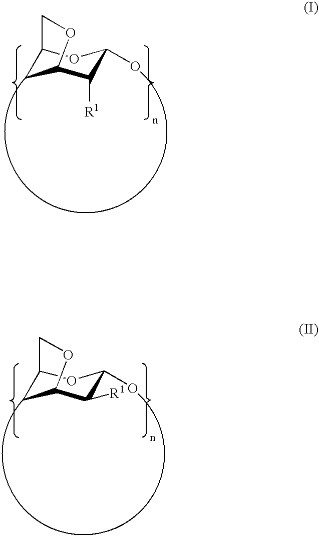
in which:
at least one of the groups R¹ represents a group —OCONHR² and the other groups R¹, which may be identical or different, represent a group corresponding to one of the formulae: —OCONHR², —OH, —OR³, —SH, —SR³, —OCOR³, —NH₂, —NHR³, —NR³R⁴, —CONH₂, —CONHR³, —CONR³R⁴, —CN, —COOR³, —OCH₂CO₂H, —COOH and —R³, in which the group(s) R², which are identical or different, represent a saturated or unsaturated aliphatic group, R³ and R⁴, which are identical or different, represent a saturated or unsaturated, aliphatic or aromatic hydrocarbon group optionally substituted with halogen atoms which may contain one or more heteroatoms chosen from O, S and N, and/or
at least one of the groups R¹ represents a group —OCONH(CR⁵R⁶)ₘNHCOOR⁷, the other groups R¹ corresponding to the same definition as that given above, R⁵ and R⁶, which are identical or different, represent H or a saturated or unsaturated aliphatic group, and R⁷ represents a glucosidic or maltosidic unit of the peranhydrocyclodextrin and m is an integer ranging from 1 to 20;
n is equal to 6, 7 or 8.

In the cyclodextrin derivative of formula (I) or (II), the aliphatic or aromatic hydrocarbon groups capable of being used for R³ and R⁴ may be of various types. They consist of a hydrocarbon chain in which some carbon atoms may be replaced with one or more heteroatoms such as O, S and N, and they may contain one or more ethylenic or acetylenic unsaturations. Moreover, the hydrocarbon group may be substituted with halogen atoms. When R³ and R⁴ are aliphatic hydrocarbon groups, they may in particular represent a linear or branched alkyl group of 1 to 20 carbon atoms, such as a methyl, ethyl, n-propyl or i-propyl group. When R³ and R⁴ are aromatic hydrocarbon groups, they may in particular represent the phenyl group or the tosyl group, optionally substituted, for example, with alkyl groups of 1 to 20 carbon atoms.

In the peranhydrocyclodextrin derivative of formula (I) or (II), when at least one of the groups R¹ represents the group —OCONHR², the group(s) R², which are identical or different, (the groups R² when several groups R¹ represent OCONHR²), represent a saturated or unsaturated aliphatic chain, that is to say an alicyclic chain which may optionally contain unsaturations. In particular, the group(s) R² may represent a linear or branched alkyl group comprising from 1 to 10 carbon atoms, such as a methyl, ethyl or hexyl group.

In the peranhydrocyclodextrin derivative of formula (I) or (II), when at least one of the groups R¹ represents the group —OCONH (CR⁵R⁶)ₘNHCOOR⁷, the groups R⁵ and R⁶, which are identical or different, represent H or a saturated or unsaturated aliphatic group, that is to say an alicyclic chain which may optionally contain unsaturations. In particular, the groups R⁵ and R⁶ may represent a linear or branched alkyl group containing from 1 to 10 carbon atoms, such as a methyl or ethyl group. In accordance with the invention, the group —OCONH(CR⁵R⁶)ₘNHCOOR⁷ serves to join two glucosidic units (if the cyclodextrin corresponds to formula (I)) or maltosidic units (if the cyclodextrin corresponds to formula (II)) of a same peranhydrocyclodextrin derivative, R⁷ thus corresponding to a glucosidic or maltosidic unit of the same peranhydrocyclodextrin derivative according to the invention.

According to a preferred embodiment of the invention, the per(3,6-anhydro)cyclodextrin derivative is an α-cyclodextrin derivative, that is to say that, in formulae (I) and (II) given above, n is equal to 6.

Preferably still, the derivative used corresponds to formula (I) or (II), in which all the groups R¹ represent the group —OCONHR² with R² having the meaning given above, and n is equal to 6. In particular, all the groups R² may represent an ethyl or hexyl radical.

Generally, the cyclodextrin derivatives of the invention may be prepared in the following manner.

When the cyclodextrin derivative corresponds to formula (I) or (II) given above, in which at least one of the groups R¹ represents the group —O—CO—NHR² and/or —OCONH(CR⁵R⁶)ₘNHCOOR⁷, the other possible groups R¹ representing a group such as those proposed above and n being equal to 6, 7 or 8, and m is an integer ranging from 1 to 20, it may be prepared by a method comprising successively:
a step consisting in reacting a per(3,6-anhydro)cyclodextrin corresponding to one of the following formulae (III) or (IV):

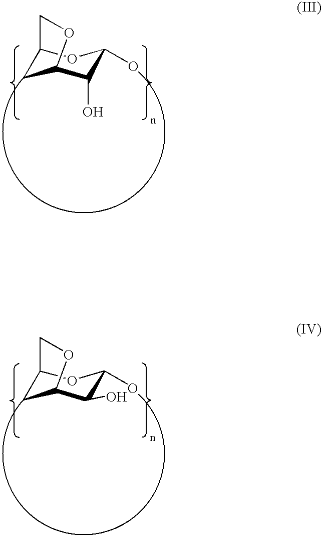

in which n is equal to 6, 7 or 8, with an isocyanate of formula OCN—R² and/or a diisocyanate OCN(CR⁵R⁶)ₘNCO in a quantity such that at least one of the OH groups represented on formulae (III) or (IV) is converted to a group —OCONHR² and/or to a group —OCONH(CR⁵R⁶)ₘNHCOOR⁷; and
a step consisting, when not all the OH groups have been converted to a group —OCONHR² and/or —OCONH(CR⁵R⁶)ₘNHCOOR⁷, in optionally reacting the remaining —OH groups with one or more reagents in order to convert them to the desired groups R¹ different from —OCONHR² and/or —OCONH(CR⁵R⁶)ₘNHCOOR⁷.

When the cyclodextrin derivative corresponds to formula (I) or (II) given above, in which the other groups R¹ represent —OR³ with R³ having the meaning given above, the procedure is carried out by reacting the partially modified cyclodextrin, obtained after the first step mentioned above, with an alkali metal hydride in order to convert the —OH group(s) to OM groups with M representing an alkali metal and then the derivative obtained is reacted with a halide of formula R³X in which R³ has the meaning given above and X is a good leaving group such as a halogen atom.

When the cyclodextrin derivative corresponds to formula (I) or (II) given above, in which the other groups R¹ represent —OCOR³, the procedure is carried out, in a first step, as above (i.e reaction of the partially modified cyclodextrine with an alkali metal hydride), and the derivative obtained is then reacted with an acid halide or anhydride of formula R³COX or (R³CO)₂O in which R³ has the meaning given above and X represent a leaving group.

When the cyclodextrin derivative corresponds to formula (I) or (II) given above, in which the other groups R¹ represent —O—CH₂—CO₂H, the procedure is carried out, in a first step, as above, (i.e reaction of the partially modified cyclodextrine with an alkali metal hydride) and the derivative obtained is then reacted with a halide of formula X—CH₂—CO₂R₈, in order to obtain a group —O—CH₂—CO₂R₈, in which X represents a halogen atom and R₈ represents H, Si(CH₃)₃ or an alkali metal. The peranhydrocyclodextrin derivative obtained is then optionally treated (when R⁸ is different from H) with an alcohol, a slightly acidic medium or water in order to convert the groups —O—CH₂—CO₂—R₈ to a group —O—CH₂—CO₂H.

When it is desired to prepare a cyclodextrin derivative in which the other group(s) R¹ represent a group of formula —SH, —SR³, —NH₂, —NHR³, —NR³R⁴, —CONR³R⁴, —CONHR³, —CONH₂, —CN, —COOR³, —COOH, or —R³, with R³ and R⁴ having the meanings given above, and n is equal to 6, 7 or 8, it is possible to carry out the following steps by starting with a partially modified peranhydrocyclodextrin, that is to say in which at least one of the groups R¹ represents —OCONHR² and/or —OCONH(CR⁵R⁶)ₘNHCOOR⁷ and the other groups R¹ represent —OH, and by carrying out the following steps:
1) reacting this peranhydrocyclodextrin with an alkali metal hydride in order to convert the —OH group(s) to —OM group(s) with M representing an alkali metal;
2) reacting the modified peranhydrocyclodextrin obtained in 1) with a chloride of formula ClSO₂R³ with R³ having the meaning given above, in order to obtain the derivative of formula (I) or (II) in which at least one of the groups R¹ is a group of formula —OSO₂R³; and
3) reacting the derivative obtained in the second step with one or more reagents appropriate for replacing —OSO₂R³ with the desired group R¹.

In this method, the partially modified per(3,6-anhydro)cyclodextrin is first of all converted to an alcoholate by the action of an alkali metal hydride, and then this alcoholate is converted to a derivative containing a leaving group of formula —OSO₂R³, which is then reacted in one or more steps with one or more reagents appropriate for replacing this leaving group with the desired group R¹.

Thus, in the case where R¹ must represent —NH₂, it is possible to react N₃M with the compound defined in 2). The compound thus obtained, called azide, can undergo a catalytic hydrogenation or can be treated in the presence of ammonia NH₃, in order to obtain the product where R¹ must represent —NH₂.

The product where R¹ must represent —NHR³ or —NR³R⁴ is obtained by reacting the compound defined in 2) with the compound NH₂R³ or NHR³R⁴.

In the case where R¹ must represent —SH or —SR³, it is possible to react the compound defined in 2) with a halide X⁻, which gives the compound with (R¹═X), which is then reacted with HS⁻ or R³S⁻ in order to give a compound where R¹ will represent SH or SR³.

When R¹ must represent a hydrocarbon group —R³, a reaction with R₂³LiCu is caused to occur in order to give a final compound where R³ then represents a hydrocarbon group, as defined above.

Likewise, the compound where R¹ represents a halogen resulting from the reaction of the compound obtained in 2) with a halide X⁻ can react with CN⁻ in order to give a final compound where R¹ will represent —CN.

Likewise, the compound where R¹ represents —CN can, by controlled hydrolysis, give a compound where R¹ will represent —CONH₂. The compound where R¹ represents —CN can, by complete hydrolysis, give a compound where R¹ will represent —COOH.

The compound where R1 represents —COOH can, by esterification, with a compound R³OH give a compound where R¹ will represent —COOR³.

The compound where R¹ represents —COOH can react with —NHR³R⁴ or NH₂R³ in the presence of DCC (dicyclohexylcarbodiimide) to give a compound where R¹ will represent —CONR³R⁴ or —CONHR³.

The subject of the present invention is also a polymer obtained by reacting at least two per(3,6-anhydro)cyclodextrins of the following formulae (III) or (IV):

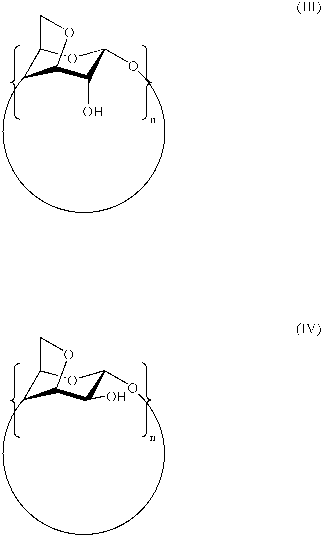

and a diisocyanate of formula OCN—(CR⁵R⁶)ₘ—NCO, in which R⁵ and R⁶, which are identical or different, represent H or a saturated or unsaturated aliphatic group, the OH groups having not reacted during the reaction to be optionally converted into groups, which are identical or different, representing groups chosen from: —OCONHR², —OR³, —SH, —SR³, —OCOR³, —NH₂, —NHR³, —NR³R⁴, —CONH₂, —CONHR³, —CONR³R⁴, —CN, —COOR³, —OCH₂COOH, —COOH and —R³, in which the group(s) R² represent a saturated or unsaturated aliphatic group, R³ and R⁴, which may be identical or different, represent a saturated or unsaturated, aliphatic or aromatic hydrocarbon group optionally substituted with halogen atoms which may contain one or more heteroatoms chosen from O, S and N, and n is equal to 6, 7 or 8 and m is an integer ranging from 1 to 20.

In this polymer, at least two per(3,6-anhydro)cyclodextrin derivatives are linked by at least one carbamate bond of the —O—CO—NH(CR⁵R⁶)ₘNH—CO—O— type, this bond being formed by reaction of two —OH groups at the 2-position of a glucosidic entity or maltositic entity of two per(3,6-anhydro)cyclodextrins. This polymer may also contain —O—CO—NH(CR⁵R⁶)ₘNH—CO—O— bonds formed by reaction of the diisocyanate mentioned above with two —OH groups of two glucosidic units or maltosidic units of the same peranhydrocyclodextrin. Finally, this polymer may also comprise —O—CO—NH(CR⁵R⁶)ₘN═C═O bonds, one end having reacted with an —OH of a cyclodextrin unit, the other end not having reacted.

In this polymer, the groups R⁵ and R⁶, which are identical or different, may represent hydrogen or a saturated or unsaturated aliphatic group.

In particular, the groups R⁵ and R⁶ may represent a linear or branched alkyl group containing from 1 to 10 carbon atoms, such as a methyl or ethyl group.

In this polymer, the aliphatic or aromatic hydrocarbon groups which can be used for R³ and R⁴ may be of various types. They consist of a hydrocarbon chain in which some carbon atoms may be replaced with one or more heteroatoms such as O, S and N and they may contain one or more unsaturations, for example ethylenic or acetylenic unsaturations. Moreover, the hydrocarbon group may be substituted with halogen atoms. When R³ and R⁴ are aliphatic hydrocarbon groups, they may in particular represent a linear or branched alkyl group of 1 to 20 carbon atoms. When R³ and R⁴ are aromatic hydrocarbon groups, they may in particular represent the phenyl group or the tosyl group, optionally substituted, for example, with alkyl groups of 1 to 20 carbon atoms.

In this polymer, when at least one of the groups R¹ represents the group —OCONHR², the group(s) R² represents a saturated or unsaturated aliphatic chain. In particular, R² may represent a linear or branched alkyl group comprising from 1 to 10 carbon atoms, such as a methyl, ethyl or hexyl group.

In the same manner as for the compounds of formula (I) and (II), the cyclodextrin units linked in the polymer described above comprise at least, partially, at the 2-positions of the anhydroglucose rings (if the polymer is obtained from the cyclodextrins of formula (III)) or anhydromaltose rings (if the polymer is obtained from the cyclodextrins of formula (IV)), bonds of the carbamate type.

Preferably, the subscript n in this polymer is equal to 6 and R⁵ and R⁶ both represent H and m is equal to 6.

Preferably still, in this polymer, all the —OH groups react with the diisocyanate mentioned above to give a carbamate bond.

The applicant has discovered, surprisingly, that, by virtue of the presence at the 2-position of groups comprising at least one carbamate functional group 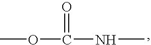
the per(3,6-anhydro)cyclodextrin derivatives described above and the per(3,6-anhydro)cyclodextrin polymers may be used in particular for the binding and separation of ions, more particularly of anions based on chromium and/or manganese.

Accordingly, the subject of the invention is also a method for binding and separating ions, comprising the steps consisting in:
bringing a medium containing the said ions into contact with:
1) a per(3,6-anhydro)cyclodextrin derivative corresponding to one of the following formulae (I) or (II): 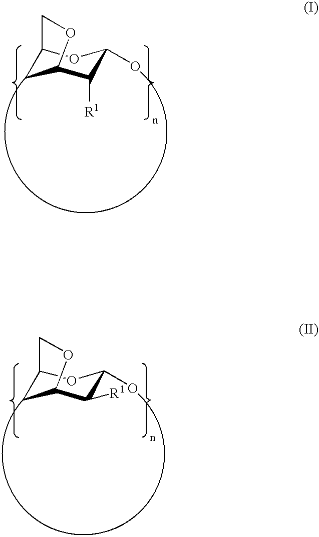
   in which:
at least one of the groups R¹represents a group —OCONHR² and the other groups R¹, which may be identical or different, represent a group corresponding to one of the formulae: —OCONHR², —OH, —OR³, —SH, —SR³, —OCOR³, —NH₂, —NHR³, —NR³R⁴, —CONH₂, —CONHR³, —CONR³R⁴, —CN, —COOR³, —OCH₂CO₂H, —COOH and —R³, in which the group(s) R², which are identical or different, represent a saturated or unsaturated aliphatic group, R³ and R⁴, which are identical or different, represent a saturated or unsaturated, aliphatic or aromatic hydrocarbon group optionally substituted with halogen atoms which may contain one or more heteroatoms chosen from O, S and N, and/or
at least one of the groups R¹ represents a group —OCONH(CR⁵R⁶)ₘNHCOOR⁷, the other groups R¹ corresponding to the same definition as that given above, R⁵ and R⁶, which are identical or different, represent H or a saturated or unsaturated aliphatic group, and R⁷ represents a glucosidic or maltosidic unit of the peranhydrocyclodextrin and m is an integer ranging from 1 to 20;
n is equal to 6, 7 or 8, and/or
2) a polymer obtained by reacting at least two per(3,6-anhydro)cyclodextrins of formula (III) or (IV), as mentioned above, and a diisocyanate of formula OCN—(CR⁵R⁶)ₘ—NCO, for which R⁵ and R⁶, which are identical or different, represent H or a saturated or unsaturated aliphatic group, the OH groups having not reacted during the reaction to be optionally converted into groups, which are identical or different, representing groups chosen from: —OCONHR², —OR³, —SH, —SR³, —OCOR³, —NH₂, —NHR³, —NR³R⁴, —CONH₂, —CONHR³, —CONR³R⁴, —CN, —COOR³, —OCH₂CO₂H, —COOH and —R³, in which the group(s) R² represents a saturated or unsaturated aliphatic group, R³ and R⁴, which may be identical or different, represent a saturated or unsaturated, aliphatic or aromatic hydrocarbon group which may contain one or more heteroatoms chosen from O, S and N, and n is equal to 6, 7 or 8 and m is an integer ranging from 1 to 20, in order to bind the said ions in the form of a complex with the per(3,6-anhydro)cyclodextrin derivative or the polymer; and
separating the said ions thus complexed from the said medium.

The ions which can be bound or separated by the method of the invention may be of various types, such as the ions of polluting metals.

However, the method of the invention applies in particular to the binding of anions based on chromium, in particular the anions containing chromium having a valency VI such as chromate or bichromate ions, and the anions based on manganese such as the permanganate ions in the form of a complex with the per(3,6-anhydro)cyclodextrin derivative or polymers mentioned above.

Indeed, toxicological studies have made it possible to demonstrate that chromium salts having a valency VI, such as the chromate ions CrO₄²⁻ or the bichromate ions Cr₂O₇²⁻, exhibit a very high toxicity to humans and to animals.

Thus, chromic acid H₂CrO₄ and its salts, which are soluble in particular in the gastric juice, can cause dermatoses and ulcerations in individuals handling them.

Potassium dichromate can prove deadly at doses of 0.25 to 0.30 g and can cause gastric disorders and enteritis.

Chromate or bichromate ions are also found to be methaemoglobinizing poisons.

According to the invention, it has been found that the cyclodextrin derivatives and the per(3,6-anhydro)cyclodextrin polymers, the said cyclodextrin corresponding to the formulae (I) and (II) given above, exhibit a high specificity for anions based on chromium or manganese because they exhibit for these metals a complexation capacity with very high yields.

In particular, a per(3,6-anhydro)cyclodextrin derivative corresponding to formula (I), which is effective for carrying out this method, is the derivative for which all the groups R¹ represent OCONHR², R² having the same definition as that given above and n is equal to 6. More precisely, R² may represent a hexyl or ethyl radical.

A polymer in accordance with the present invention, which may be effectively used in the context of the binding method, is a polymer for which n is equal to 6, R⁵ and R⁶ both represent H and m is equal to 6.

By virtue of these compounds, it is possible to separate the anions based on chromium and manganese from the surrounding medium in the form of a complex.

Accordingly, the subject of the invention is also the complexes of an ion chosen from CrO₄²⁻, Cr₂O₇²⁻ and MnO₄⁻ with a per(3,6-anhydro)cyclodextrin derivative of formula (I) or (II) described above and/or with a polymer as defined above.

Preferably, the complex of an ion chosen from CrO₄²⁻, Cr₂O₇²⁻ and MnO₄⁻, when the peranhydrocyclodextrin derivative corresponds to formula (I), is such that all the groups R¹ represent the group —O—CO—NHR² and n is equal to 6, R² having the same definition as that given above.

To carry out the method of separating ions of the invention, it is possible to use the per(3,6-anhydro)cyclodextrin derivative of formula (I) or (II) or the per(3,6-anhydro)cyclodextrin and diisocyanate polymers described above in the form of an aqueous solution or an organic solution.

When the medium containing the ions to be separated or to be bound is an aqueous solution, it is possible to dissolve the cyclodextrin derivative in an organic solvent which is immiscible with the aqueous solution in order to form the complex in the organic solution and to easily separate it from the aqueous solution, for example by mere decantation followed by separation of the aqueous solution and of the immiscible organic solvent containing the complexed ions.

It is also possible to use the cyclodextrin derivative or the polymers in aqueous solution, in particular to bring about decontamination of human beings or alternatively as a preparation in dressings (agarose gels).

Indeed, these compounds are biocompatible and can therefore be administered to humans and to animals to bring about the binding of chromium and manganese in the form of a complex and to thereby avoid their interaction with the organs of the human or animal body.

Accordingly, the subject of the invention is also a pharmaceutical composition for the decontamination, in relation to chromium and to manganese, of a human being, characterized in that it comprises a per(3,6-anhydro)cyclodextrin derivative corresponding to one of formulae (I) and (II) defined above and/or a per(3,6-anhydro)cyclodextrin and diisocyanate polymer as described above.

Preferably, the derivative used in this composition is such that all the groups R¹ represent the group —O—CO—NHR² and n is equal to 6, R² having the same definition as that given above.

This composition may be administered by the oral route or by injection. Administered by the oral route, it should be packaged in a suitable manner so as to go through the stomach without being hydrolyzed.

Other characteristics and advantages of the invention will appear more clearly on reading the examples which follow, which are given by way of illustration and without limitation, with reference to the accompanying drawings.

### BRIEF DESCRIPTION OF THE FIGURES

FIG. 1 is a schematic representation of the retardation factors Rf of various anions, with the per(3,6-anhydro)cyclodextrin of Example 1.

FIG. 2 is a schematic representation of the retardation factors Rf of various cations, with the per(3,6-anhydro)cyclodextrin derivative of Example 1.

### DETAILED DESCRIPTION OF SPECIFIC EMBODIMENTS

### EXAMPLE 1

### Preparation of per(3,6-anhydro)cyclomaltohexaose per-2-O-ethylcarbamate

This compound corresponds to formula (I) given above in which all the groups R¹ represent —OCONHCH₂CH₃.

1 g of per(3,6-anhydro)cyclomaltohexaose, dried under a vacuum produced by a slide vane rotary vacuum pump for 2 hours, are weighed out and there are added 50 ml of anhydrous dimethyl sulphoxide (DMSO) and 3 ml of ethyl isocyanate in the cold state. The solution is heated at 100° C. overnight. The solution is then cooled and 1.5 ml of ethyl isocyanate are added and the medium is re-heated to 100° C.

After leaving overnight, the solution is cooled and then treated with 10 ml of methanol and kept stirring for 1 hour. The solution is then dried using a rotary evaporator followed by a slide vane rotary vacuum pump.

The residue obtained is then passed over a silica gel column (eluent methanol/water 1:6).

This product can be used as it is for the complexing of chromium or manganese.

### EXAMPLE 2

### Preparation of per(3,6-anhydro)cyclomaltohexaose per-2-O-hexylcarbamate

This compound corresponds to formula (I) given above in which all the groups R¹ correspond to —OCONH(CH₂)₅—CH₃ and n is equal to 6.

444 mg of per(3,6-anhydro)cyclomaltohexaose are weighed after drying for 3 hours under a slide vane rotary vacuum pump, and 25 mL of dimethyl sulphoxide (DMSO) are added under argon, followed by 1.5 mL of hexyl isocyanate. The solution is heated to 70° C., with magnetic stirring. After heating overnight at 70° C., the cooled solution is supplemented with 0.8 mL of hexyl isocyanate and is kept at 10° C. overnight. Next, the solution is cooled and supplemented with methanol, with stirring. After stirring for 1 hour, the solvents are removed and the residue is purified by column chromatography (silica gel, methanol/chloroform 1:6).

### EXAMPLE 3

### Demonstration of the Complexing of Anions by the Compound of Example 1, by Ion-Exchange Plate Chromatography

The use of thin layer chromatography plates charged with ions allows rapid evaluation of the complexing of these ions by the species to be evaluated; in the present case, plates of the Polygram Ionex 25-SA-Na type (Macherey-Nagel, ref.: 80613) charged with various counterions to be evaluated.

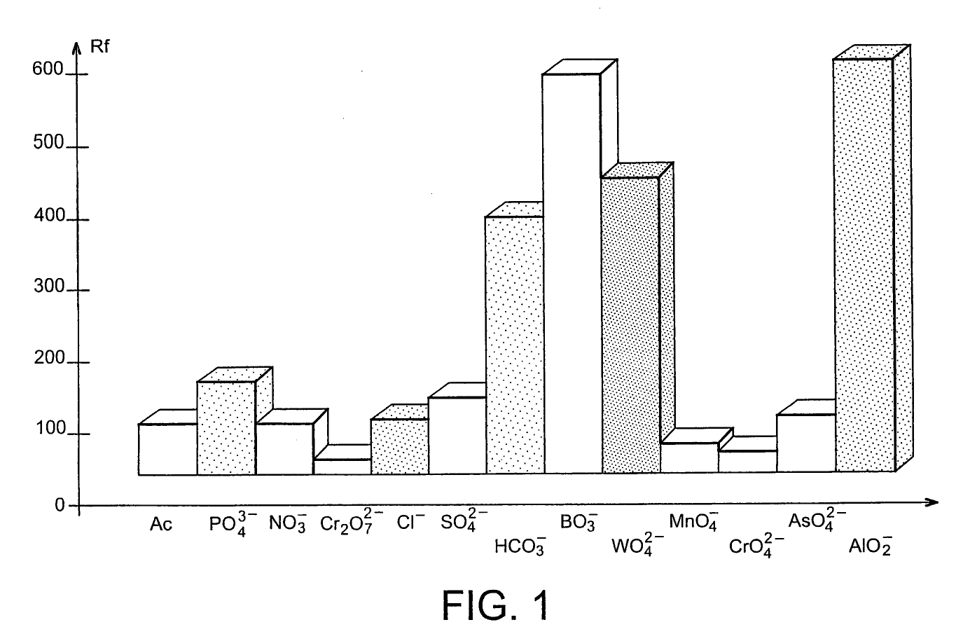

Thus, chromatography plates are used onto which are respectively bound the ions acetyl COO⁻ (designated Ac in FIG. 1), PO₄³⁻, NO₃⁻, Cr₂O₇²⁻, Cl⁻, SO₄²⁻, HCO₃⁻, BO₃⁻, WO₄²⁻, MnO₄⁻, CrO₄²⁻, AsO₄²⁻, AlO₂⁻.

In each test, there is introduced onto the plate the compound of Example 1 which will be retained on the plate if it complexes the ions. The plates are then developed four times in water, because of the low solubility in water, and then the retardation factor Rf, which corresponds to the ratio (distance covered by the cyclodextrin derivative/distance covered by the solvent), is determined. The lower the Rf, for a given ion, the more the ion in question will form a complex with the cyclodextrin compound.

The results obtained are represented in FIG. 1.

It is thus observed that the cyclodextrin prepared according to Example 1 has a high complexing rate for the chromium ions such as the ions Cr₂O₇²⁻ and CrO₄²⁻ and the manganese ions MnO₄⁻.

Because of this, the cyclodextrin compounds according to the invention, and in particular that prepared according to Example 1, are particularly advantageous in the field of environmental contamination and in the field of human decontamination in relation to anions based on chromium and manganese.

Likewise, tests were carried out to see whether the derivatives according to the invention were capable of complexing cations. Tests were carried out with the following ions:

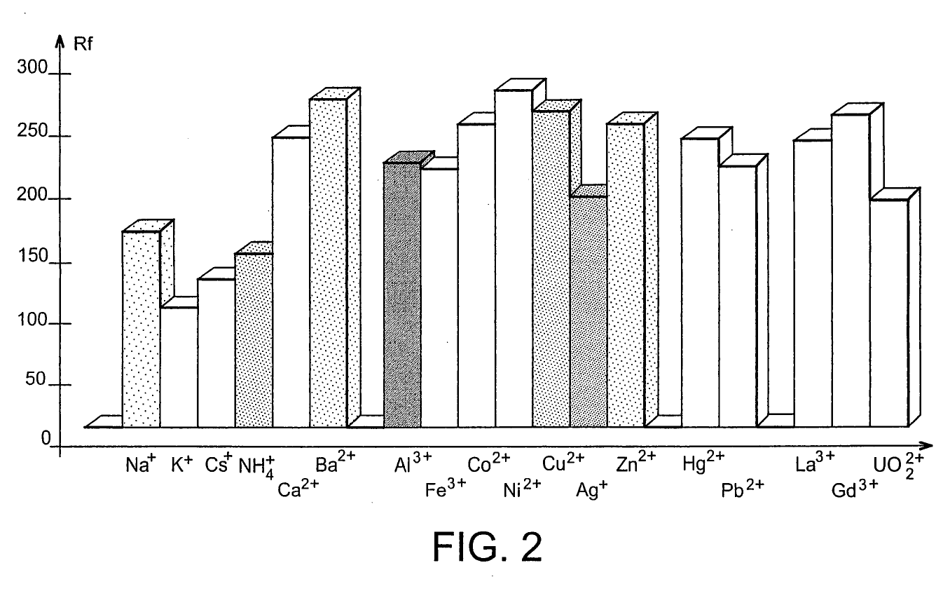

Na⁺, K⁺, Cs⁺,NH₄⁺,Ca²⁺,Ba²⁺, Al³⁺, Fe³⁺, Co²⁺, Ni²⁺, Cu²⁺, Ag²⁺, Zn²⁺, Hg²⁺, Pb²⁺, La³⁺, Gd³⁺, UO₂²⁺. According to the results assembled in FIG. 2, none of these cations efficiently forms a complex with the peranhydrocyclodextrin prepared according to Example 1.

### EXAMPLE 4

### Preparation of a Polymer Obtained by Reacting a per(3,6-anhydro)cyclomaltohexaose and hexyl diisocyanate

5 ml of anhydrous dimethylformamide (DMF) and 0.934 mL of hexyl diisocyanate (2 equivalents per 1 mmol of anhydro) are added to 2.5 g of per(3,6-anhydro)cyclomaltohexaose dried using a pump for 2 hours. The medium is heated to 90° C., with stirring, and left to react overnight. The product is then supplemented with methanol (20 ml) and left to react for 1 hour. A powder is recovered by scraping, it is centrifuged and then dried. 3.21 g of polymer are recovered after air-drying at room temperature. This polymer is characterized by its microanalysis and by NMR on the solid.

### EXAMPLE 5

### Preparation of Polymer Complexes

820 mg of polymer prepared in Example 4 are weighed, and a solution containing 312 mg of potassium dichromate and 10 mL of water is added thereto. The product is left overnight, with stirring. It is then centrifuged and washed with 50 mL of water and recentrifuged. The latter operation is repeated four times. The product recovered (800 mg) is analyzed by microanalysis and NMR on the solid. It is observed that the chromium is complexed in its bichromate and chromate form. The microanalysis shows that one site out of two is occupied by the chromium and that the product is not oxidized over time.

### COMPARATIVE EXAMPLE

### Preparation of Complexes Based on Inositol

5 g of myoinositol are weighed and they are then dried using a slide vane rotary vacuum pump for 2 hours. 40 ml of dimethyl sulphoxide (DMSO) and 4.85 mL of hexyl diisocyanate are then added, with stirring. The reaction mixture is kept at 100° C. overnight. After cooling, the solution is then supplemented with 10 ml of methanol. After 1 hour, the whole is dried, precipitated from water and centrifuged.

500 mg of this polymer are then treated with an aqueous solution of potassium dichromate (500 mg, 10 mL). After leaving overnight, with stirring, at room temperature, the product is separated by decantation and taken up in water (50 mL), stirred for 1 hour and centrifuged 4 times. The residue weighed after air-drying (516 mg) is studied by NMR on the solid and sent for microanalysis. It was possible to observe that the chromium complexing rate is of the order of 8%, which is very low compared with the products of the invention. It was also possible to demonstrate that the chromium complexing the product has the valency 3. Despite the presence of residual alcohol groups, the product can be stable and can be optionally recycled.

### LIST OF THE DOCUMENTS CITED

[1]: D. Duchêne “Pharmaceutical application of cyclodextrins” in “Cyclodextrins and their industrial uses”. D. Duchêne Ed., Editions de Santé, Paris, 1987, pages 213-257.
[2]: Gadelle A. and Defaye J., Angew. Chem. Int. Ed. Engl., (1991), 30, pages 78-79.
[3]: Ashton P. R., Ellwood P., Staton I. and Stoddart J. F., Angew. Chem. Int. ed. Engl., (1991), 30, pages 80-81.
[4]: Yamamura H. and Fujita K. Chem. Pharm. Bull., (1991) 39, pages 2505-2508.
[5]: Yamamura H., Ezuka T., Kawase Y., Kawai M., Butsugan Y. and Fujita K., J. Chem. Soc., Chem. Com., (1993), pages 636-637.
[6]: Yamamura H. Nagaoka H., Kawai M. and Butsugan Y., Tetrahedron Lett. (1995) 36, pages 1093-1094.
[7]: FR-A 2 744 124.
[8]: FR-A 2 764 525.
[9]: FR-A 2 807 044.

1. Per(3,6-anhydro)cyclodextrin derivative corresponding to one of the following formulae: 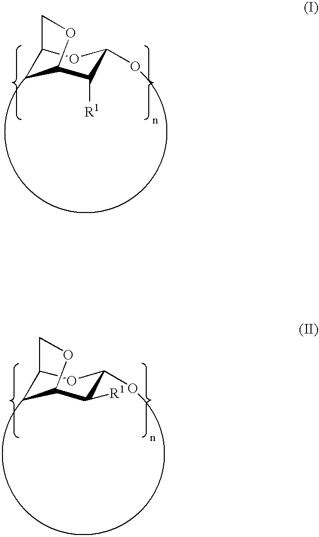
in which:
at least one of the groups R¹ represents a group —OCONHR² and the other groups R¹, which may be identical or different, represent a group corresponding to one of the formulae: —OCONHR², —OH, —OR³, —SH, —SR³, —OCOR³, —NH₂, —NHR³, —NR³R⁴, —CONH₂, —CONHR³, —CONR³R⁴, —CN, —COOR³, —OCH₂CO₂H, —COOH and —R³, in which the group(s) R², which are identical or different, represent a saturated or unsaturated aliphatic group, R³ and R⁴, which are identical or different, represent a saturated or unsaturated, aliphatic or aromatic hydrocarbon group optionally substituted with halogen atoms which may contain one or more heteroatoms chosen from O, S and N, and/or
at least one of the groups R¹ represents a group —OCONH(CR⁵R⁶)ₘNHCOOR⁷, the other groups R¹ corresponding to the same definition as that given above, R⁵ and R⁶, which are identical or different, represent H or a saturated or unsaturated aliphatic group, and R⁷ represents a glucosidic or maltosidic unit of the peranhydrocyclodextrin and m is an integer ranging from 1 to 20;
n is equal to 6, 7 or 8.

2. Per(3,6-anhydro)cyclodextrin derivative according to claim 1, in which all the groups R¹ represent the group —OCONHR² with R² having the same meaning as in claim 1, and n is equal to 6.

3. Per(3,6-anhydro)cyclodextrin derivative according to claim 2, in which R² represents an ethyl radical.

4. Per(3,6-anhydro)cyclodextrin derivative according to claim 2, in which R² represents a hexyl radical.

5. Method for preparing a per(3,6-anhydro)cyclodextrin derivative corresponding to one of the following formulae (I) and (II): 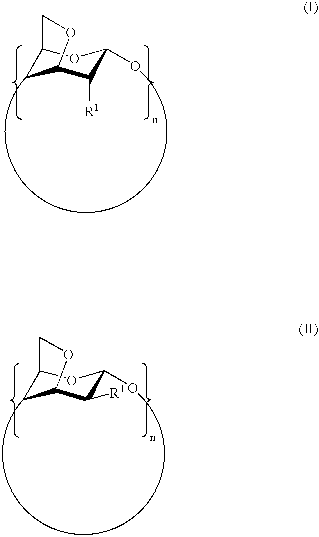
in which:
at least one of the groups R¹ represents a group —OCONHR² and the other groups R¹, which may be identical or different, represent a group corresponding to one of the formulae: —OCONHR², —OH, —OR³, —SH, —SR³, —OCOR³, —NH₂, —NHR³, —NR³R⁴, —CONH₂, —CONHR³, —CONR³R⁴, —CN, —COOR³, —OCH₂CO₂H, —COOH and —R³, in which the R² group(s), which are identical or different, represent a saturated or unsaturated aliphatic group, R³ and R⁴, which are identical or different, represent a saturated or unsaturated, aliphatic or aromatic hydrocarbon group optionally substituted with halogen atoms which may contain one or more heteroatoms chosen from O, S and N, and/or
at least one of the groups R¹ represents a group —OCONH(CR⁵R⁶)ₘNHCOOR⁷, the other groups R¹ corresponding to the same definition as that given above, R⁵ and R⁶, which are identical or different, represent H or a saturated or unsaturated aliphatic group, and R⁷ represents a glucosidic or maltosidic unit of the peranhydrocyclodextrin and m is an integer ranging from 1 to 20;
n is equal to 6, 7 or 8, said process comprising successively:
a step consisting in reacting a per(3,6-anhydro)cyclodextrin corresponding to one of the following formulae (III) or (IV): 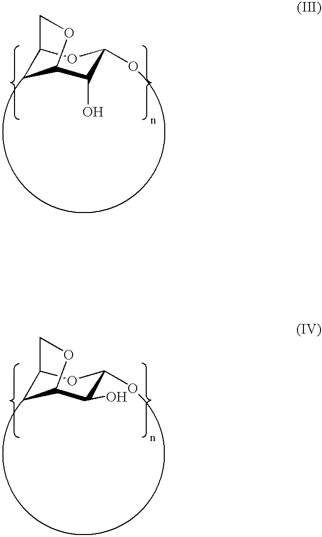
in which n is equal to 6, 7 or 8, with an isocyanate of formula OCN—R² and/or a diisocyanate OCN(CR⁵R⁶)ₘNCO in a quantity such that at least one of the OH groups is converted to a group —OCONHR² and/or to a group —OCONH(CR⁵R⁶)ₘNHCOOR⁷; and
a step consisting, when not all the OH groups have been converted to a group —OCONHR² and/or —OCONH(CR⁵R⁶)ₘNHCOOR⁷, in optionally reacting the remaining OH groups with one or more reagents in order to convert them to the desired groups R¹ different from —OCONHR² and/or —OCONH(CR⁵R⁶)ₘNHCOOR⁷.

6. Polymer obtained by reacting at least two per(3,6-anhydro)cyclodextrins corresponding to one of the following formulae (III) or (IV): 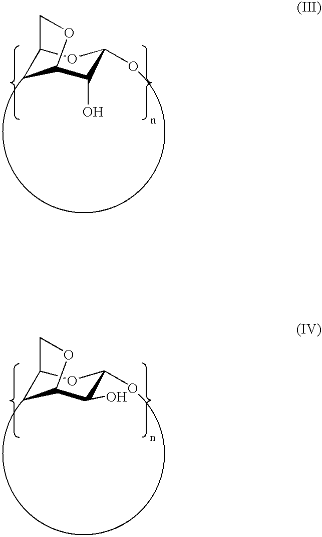
in which n is equal to 6, 7 or 8 and a diisocyanate of formula OCN—(CR⁵R⁶)ₘ—NCO, in which R⁵ and R⁶, which are identical or different, represent H or a saturated or unsaturated aliphatic group and m is an integer ranging from 1 to 20, the OH groups having not reacted during the reaction to be optionally converted into groups, which are identical or different, representing groups chosen from: —OCONHR², —OR³, —SH, —SR³, —OCOR³, —NH₂, —NHR³, —NR³R⁴, —CONH₂, —CONHR³, —CONR³R⁴, —CN, —COOR³, —OCH₂COOH, —COOH and —R³, in which the group(s) R² represent a saturated or unsaturated aliphatic group, R³ and R⁴, which may be identical or different, represent a saturated or unsaturated, aliphatic or aromatic hydrocarbon group optionally substituted with halogen atoms which may contain one or more heteroatoms chosen from O, S and N.

7. Polymer according to claim 6, for which n is equal to 6 and R⁵ and R⁶ both represent H and m is equal to 6.

8. Method for binding and separating ions, comprising the steps consisting in:
bringing a medium containing the said ions into contact with:
—1) a per(3,6-anhydro)cyclodextrin derivative corresponding to one of the following formulae (I) or (II): 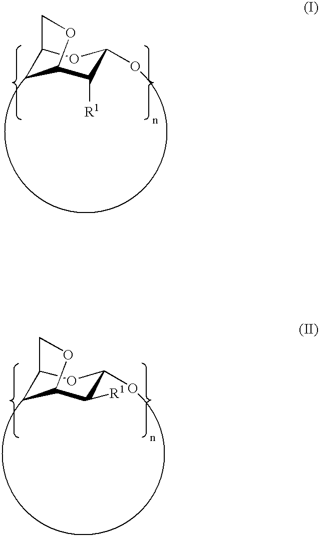
in which:
at least one of the groups R¹ represents a group —OCONHR² and the other groups R¹, which may be identical or different, represent a group corresponding to one of the formulae: —OCONHR, —OH, —OR³, —SH, —SR³, —OCOR³, —NH₂, —NHR³, —NR³R⁴, —CONH₂, —CONHR³, —CONR³R⁴, —CN, —COOR³, —OCH₂CO₂H, —COOH and —R³, in which the group(s) R², which are identical or different, represent a saturated or unsaturated aliphatic group, R³ and R⁴, which are identical or different, represent a saturated or unsaturated, aliphatic or aromatic hydrocarbon group optionally substituted with halogen atoms which may contain one or more heteroatoms chosen from O, S and N, and/or
at least one of the groups R¹ represents a group —OCONH(CR⁵R⁶)ₘNHCOOR⁷, the other groups R¹ corresponding to the same definition as that given above, R⁵ and R⁶, which are identical or different, represent H or a saturated or unsaturated aliphatic group, and R⁷ represents a glucosidic or maltosidic unit of the peranhydrocyclodextrin and m is an integer ranging from 1 to 20;
n is equal to 6, 7 or 8, and/or
2) a polymer obtained by reacting at least two per(3,6-anhydro)cyclodextrins of formula (III) or (IV), as defined in claim 6, and a diisocyanate of formula OCN—(CR⁵R⁶)ₘ—NCO, for which R⁵ and R⁶, which are identical or different, represent H or a saturated or unsaturated aliphatic group and m is an integer ranging from 1 to 20, the OH groups having not reacted during the reaction to be optionally converted into groups, which are identical or different, representing groups chosen from: —OCONHR², —OR³, —SH, —SR³, —OCOR³, —NH₂, —NHR³, —NR³R⁴, —CONH₂, —CONHR³, —CONR³R⁴, —CN, —COOR³, —OCH₂CO₂H, —COOH and —R³, in which the group(s) R², which are identical or different, represent a saturated or unsaturated aliphatic group, R³ and R⁴, which may be identical or different, represent a saturated or unsaturated, aliphatic or aromatic hydrocarbon group which may contain one or more heteroatoms chosen from O, S and N, and n is equal to 6, 7 or 8, in order to bind the said ions in the form of a complex with the per(3,6-anhydro)cyclodextrin derivative or the polymer; and
separating the said ions thus complexed from the said medium.

9. Method according to claim 8, in which the said ions are anions based on chromium or manganese.

10. Method according to claim 9, in which the per(3,6-anhydro)cyclodextrin derivative corresponds to formula (I) in which all the groups R¹ represent the group —OCONHR² with R² having the same meaning as in claim 1, and n is equal to 6.

11. Method according to claim 10, in which R² represents an ethyl or hexyl radical.

12. Method according to claim 8, in which the polymer is as defined in claim 7.

13. Method according to claim 8, in which, since the said medium is an aqueous solution, the per(3,6-anhydro)cyclodextrin derivative or the polymer is dissolved in an organic solvent which is immiscible with the said aqueous solution.

14. Pharmaceutical composition for the decontamination, in relation to ions based on chromium or manganese, of a human being, comprising:
(1) a per(3,6-anhydro)cyclodextrin derivative corresponding to one of the following formulae (I) or (II): 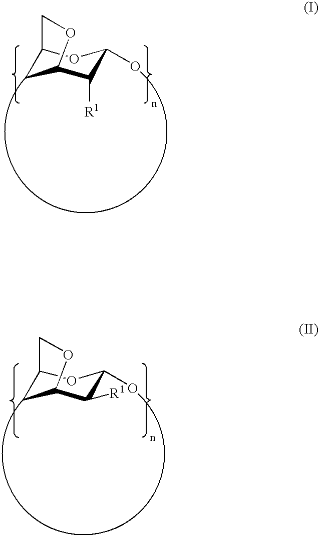
in which:
at least one of the groups R¹ represents a group —OCONHR² and the other groups R¹, which may be identical or different, represent a group corresponding to one of the formulae: —OCONHR², —OH, —OR³, —SH, —SR³, —OCOR³, —NH₂, —NHR³, —NR³R⁴, —CONH₂, —CONHR³, —CONR³R⁴, —CN, —COOR³, —OCH₂CO₂H, —COOH and —R³, in which the group(s) R², which are identical or different, represent a saturated or unsaturated aliphatic group, R³ and R⁴, which are identical or different, represent a saturated or unsaturated, aliphatic or aromatic hydrocarbon group optionally substituted with halogen atoms which may contain one or more heteroatoms chosen from O, S and N, and/or
at least one of the groups R¹ represents a group —OCONH(CR⁵R⁶)ₘNHCOOR⁷, the other groups R¹ corresponding to the same definition as that given above, R⁵ and R⁶, which are identical or different, represent H or a saturated or unsaturated aliphatic group, and R⁷ represents a glucosidic or maltosidic unit of the peranhydrocyclodextrin and m is an integer ranging from 1 to 20;
n is equal to 6, 7 or 8, and/or
(2) a polymer as defined in claims 6 and 7.

15. Pharmaceutical composition according to claim 14, in which all the groups R¹ represent the group —O—CO—NHR² and n is equal to 6, R² having the same meaning as in claim 1.

16. Complex of an ion chosen from CrO₄²⁻, Cr₂O₇²⁻ and MnO₄²⁻ with:
(1) a per(3,6-anhydro)cyclodextrin derivative corresponding to one of the following formulae: 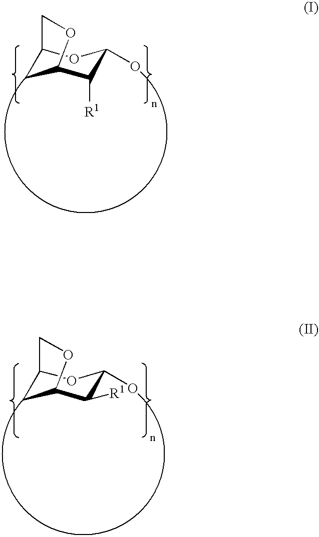
in which:
at least one of the groups R¹ represents a group —OCONHR² and the other groups R¹, which may be identical or different, represents a group corresponding to one of the formulae: —OCONHR², —OH, —OR³, —SH, —SR³, —OCOR³, —NH₂, —NHR³, —NR³R⁴, —CONH₂, —CONHR³, —CONR³R⁴, —CN, —COOR³, —OCH₂CO₂H, —COOH and —R³, in which the group(s) R², which are identical or different, represent a saturated or unsaturated aliphatic group, R³ and R⁴, which are identical or different, represent a saturated or unsaturated, aliphatic or aromatic hydrocarbon group optionally substituted with halogen atoms which may contain one or more heteroatoms chosen from O, S and N, and/or
at least one of the groups R¹ represents a group —OCONH(CR⁵R⁶)ₘNHCOOR⁷, the other groups R¹ corresponding to the same definition as that given above, R⁵ and R⁶, which are identical or different, represent H or a saturated or unsaturated aliphatic group, and R⁷ represents a glucosidic or maltosidic unit of peranhydrocyclodextrin and m is an integer ranging from 1 to 20;
n is equal to 6, 7 or 8, and/or
(2) a polymer as defined in claims 6 and 7.

17. Complex according to claim 16, in which the per(3,6-anhydro)cyclodextrin derivative corresponds to formula (I) in which all the groups R¹ represent the group —O—CO—NHR² and n is equal to 6, R² having the same meaning as in claim 1.
